SEQUENCE LISTING (4 sequences)

SEQ ID NO: 1 - DNA, 90 bp, Artificial Sequence
  Synthetic
cacacgcgcgatcaccacgcgcgtgaatacgcgcgtcccaacgcgcgacattacatcgcgcgtgaccttgacgcgcgttatgatgacccc

SEQ ID NO: 2 - DNA, 90 bp, Artificial Sequence
  Synthetic
acatcatactttccagcaacgcgcggagactcgcgcggtttaagctcgcgcgtccaaccttcaggacgcgcggggtcatcataacgcgcg

SEQ ID NO: 3 - DNA, 90 bp, Artificial Sequencce
  Synthetic
gttgctggaaagtatgatgtatatttgatcaatccccagggaaaagcctcgcgcgataaagtagaattgatcgcgcgatttgaaaaggtc

SEQ ID NO: 4 - DNA, 88 bp, Artificial Sequence
  Synthetic
cctggattcgcgcgatctcccagttcgcgcgaagtcaaaatcacgcgcgtccaatgagatacgcgcgaccttttcaaatcgcgcgatc

(19) United States
(12) Patent Application Publication
(10) Pub. No.: US 2009/0181391 A1
(43) Pub. Date: Jul. 16, 2009

(54) METHODS FOR ANALYSIS OF DNA METHYLATION PERCENTAGE

(75) Inventors: Michael Karberg, Anaheim, CA (US); Jia Xiyu, Newport Beach, CA (US)

Correspondence Address: Claypool Intellectual Property, 107 1st Ave. N 405, Seattle, WA, 98109, US

(73) Assignee: ZYMO RESEARCH CORPORATION, Orange, CA (US)

(21) Appl. No.: 12/263,465

(22) Filed: Nov. 1, 2008

Related U.S. Application Data
(60) Provisional application No. 61/001,468, filed on Nov. 1, 2007.

(51) Int. Cl. C12Q 1/68

(52) U.S. Cl. 435/6

(57) ABSTRACT

Methods are disclosed for determining the methylation state of DNA samples using melt analysis including high resolution melt analysis. Methods are also provided for determining methyltransferase activity using melt analysis including high resolution melt analysis. Additionally, kits of parts are provided.

### CROSS REFERENCE TO RELATED APPLICATIONS

This application claims priority from U.S. provisional application No. 61/001,468 of Karberg et al., entitled Total DNA Methylation Analysis, filed Nov. 1, 2007, which is hereby incorporated by reference.

### TECHNICAL FIELD

The disclosure provides methods, reagents, and kits for determining the total or global methylation status of genomic DNA using high-resolution melting analysis (HRM).

### BACKGROUND

The phenomena of DNA methylation is of increasing importance clinically and the focus of intense research efforts. It is now thought that regulation of methylation status is fluid during a person's lifetime and an indicator of health and disease with additional focus on epigenetic factors and inheritance of methylation states. DNA methylation has been shown to play a central role in gene imprinting, embryonic development, X-chromosome gene-silencing, and cell-cycle regulation. Aberrant methylation is widespread in cancer and may be among the earliest changes to occur during oncogenesis (Stirzaker. 1997). All of these observations call for increased tools to analyze genomic methylation states and assay of these may vary based on the totality of input factors and have implications for development of personalized approaches for medicine.

In many animals (and plants), DNA methylation involves the addition of a methyl group to the fifth-position carbon of the cytosine pyrimidine ring via a methyltransferase enzyme (Adams, 1995). The majority of DNA methylation in mammals occurs in 5′-CpG-3′ dinucleotides but other methylation patterns have been described. Remarkably, about 80-percent of all 5′-CpG-3′ dinucleotides found in mammalian genomes are methylated. Of the remaining 20-percent unmethylated dinucleotides, most are found within gene promoters and first exons.

Prior to this disclosure a primary method to analyze DNA methylation patterns utilized methylation specific PCR (MSP) and bisulfite/desulfonation treatment of DNA, converting methylated Cytosines (C) to Uracil (U) residues, which are only detected by select primer sets during PCR (Frommer, M. et al 1992; Herman J. G. et al 1996). Other commonly employed methods such as genomic sequencing and Pyrosequencing are used but suffer from sensitivity problems or availability of proprietary instrumentation (Frommer, M. et al. 1992; Clark S. J. et al. 1994; Colella, S. et al. 2003).

Recently, methylation-sensitive high resolution melting (MS-HRM) analysis of both methylated and non-methylated PCR fragments after bisulfite treatment of sample DNA was shown to allow sensitive estimation of an unknown methylation status by comparing the melting curves from unknown scruples to samples either fully methylated or non-methylated (Wojdacz and Dobrovic, 2007). Though promising-, this study relied on bisulfite treatment, adding complexity and processing time. The major limitation of the current methodology is that it relies on a locus by locus determination and typically requires bisulfite/desulfonation treatment for analysis.

Methods that remain sensitive, but avoid cumbersome bisulfite treatment and desulfonation steps are desirable to reduce time and guard against sample loss, thus increasing usefulness in assays on rare clinical samples. Further, a larger total or global view of a cell's, tissue's or organism's overall methylation profile is necessary to assess changes that may occur over ones lifetime. Methods to accurately assess methyltransferase activity would also facilitate methylation analysis. Advances in personalized medicine approaches could allow for adjustment in lifestyle, diet, and other factors thought to influence methylation patterns as a prophylactic to the onset of disease.

### REFERENCES

Stirzaker, C. S., Millar, D. S., Paul, C. L., Warnecke, P. M., Harrison, J., Vincent, P. C., Frommer, M. and Clark, S. J., Extensive DNA Methylation Spanning the Rb Promoter in Retinoblastoma Tumors. (1997) Cancer Research, 57:2229-2237.
Adams R. L., Eukaryotic DNA methyltransferases, structure and function. (1995) Bioessays, 17:139-145.
Woodcock, D. M., Crowther, P. J., Doherty, J., Jefferson, S. DeCruz, E., Noyer-Weidner, M., Smith, S. S. Michael, M. Z., and Graham, M. W., Quantitative evaluation of Escherichia coli host strains for tolerance to cytosine methylation in plasmid and phage recombinants. (1989) Nucleic Acids Res., 17:3469-3478.
Palmer, B. R. and Marinus, M. G., The dam and dcm strains of Escherichia coli—A review. (1994) Gene, 143:1-12.
Cihák A., Biological effects of 5-azacytidine in eukaryotes. (1974) Oncology 30:405-422.

Frommer, M., McDonald, L. E., Millar, D. S., Collis, C. M., Watt, F., Grigg, G. W., and Molloy, P. L., A genomic sequencing protocol that yields a positive display of 5-methylcytosine residues in individual DNA strands. (1992) Proc. Natl. Acad. Sci., 89:1827-1831.
Clark, S. J., Harrison, J., Paul, C. L., and Fromer, M., High sensitivity mapping of methylated cytosines. (1994) Nucleic Acids Res., 22:2990-2997.
Colella, S., Shen, L., Baggerly, K. A., Issa, J. P., and Krahe, R., Sensitive and quantitative universal Pyrosequencing methylation analysis of CpG sites. (2003) Biotechniques, 35:146-150.
Herman, J. G., Graff, J. R., Myohanen, S., Nelkin, B. D., and Baylin, S. B., Methylation-Specific PCR, a novel PCR assay for methylation status of CpG islands. (1996) Proc. Natl. Acad. Sci., 93:9321-9826.
Derringer, P. L., Approaches to rapid DNA sequence Analysis. (1983) Anal. Biochem., 135:247-263.
Bodentheich, A., Chissoe, S., Wang, Y.-F., and Roe, B. A., Shotgun cloning as the strategy of choice to generate templates for high tluhoughput dideoxynucleotide sequencing. In Automated DNA sequencing and analysis, (ed. M. D. Adams et al.), pp 42-50. Academic Press, San Diego, (1994).
Oefner, P. J., Hunicke-Smith, S. P., Chiang, L., Dietrich, F., Mulligan. J., and Davis, R. W. Efficient random subcloning of DNA sheared in a recirculating point-sink flow system. (1996) Nucleic Acids Res., 24:3879-3886.
Davidson, P. F., The effect of hydrodynamic shear on the deoxyribonucleic acid from T2 and T4 bacteriophages. (1959) Proc. Natl. Acad. Sci., 45:1560-1568.
Stewart, F. J. and Raleigh, E. A., Dependence of McrBC cleavage on distance between recognition elements. (1998) Biol. Chem., 379:611-616.
Wittwer, C. T., Herrmann, M. G., Moss, A. A., and Rasmussen, R. P., Continuous fluorescence monitoring of rapid cycle DNA amplification. (1997) Biotechniques 22:130-131.
Wittwer, C. T., Ririe, K. M., Andrew, R. V., David, D. A., Gundry, R. A., and Balis, U. J., The LightCycler: a microvolume multisample fluorimeter with rapid temperature control. (1997) Biotechniques, 22:176-181.
Ririe, K. M., Rasmussen, R. P., and Wittwer, C. T., Product differentiation by analysis of DNA melting curves during the polymerase chain reaction. (1997) Anal. Biochem., 245:154-160.
Lay, M. J. and Wittwer, C. T, Real Time fluorescent genotyping of factor V Leiden during rapid-cycle PCR. (1997) Clin. Chem., 43:2262-2267.
Reed, C. H. and Wittwer, C T, Sensitivity and specificity of single-nucleotide polymorphism scanning by high-resolution melting analysis. (2004) Clin. Chem., 50:1748-1754.
Rhee, I., Bachman, K. E., Park, B. H., Jair, K. W., Yen, R. W., Schuebel, K. E., Cui, H., Feinberg, A. P., Lengaier, C., and Kinzler, K. W. DNMT1 and DNMT3b cooperate to silence genes in human cancer cells. (2002) Nature, 416:552-556.
Chon, L. S, Lyon, E., and Wittwer, C. T., A comparison of High-resolution Melting Analysis with Denaturing High-Performance Liquid Chromatography for Mutation Scanning. (2005) Am. J. clin. Pathol., 124:330-338.
Wojdacz, T. K. and Dobrovic, A., Methylation-sensitive high resolution melting (MS-HRM): a new approach for sensitive and high-throughput assessment of methylation. (2007) Nucleic Acids Research, 35:e41.

### BRIEF DESCRIPTION OF THE DRAWINGS

FIG. 1: Depicts high resolution melt analysis of DNA samples.

FIG. 2: Depicts high resolution melting analysis of PCR products of 297 bp, methylated (100%) and non-methylated (0%).

FIG. 3: Depicts high resolution melting analysis of E. coli chromosomal DNA samples.

FIG. 4: Depicts high resolution melting analysis of inter-molecular methylation differences of human cell line derived genomic DNA, containing less than 5% native methylation, mixed with artificially-methylated genomic DNA.

FIG. 5: Depicts the predictability of high resolution melting analysis compared to the actual methylation percentage.

FIG. 6: Depicts a methyltransferase activity assay using HRM.

### SUMMARY OF THE DISCLOSURE

Methods are provided for determining the methylation state of DNA samples, including generating DNA samples and measuring the melting profiles of the DNA samples followed by comparing these melting profiles to reference DNA samples of known methylation state for a plurality of cytosine and adenosine nucleotides. The methylation state of the DNA samples is determined from the differences with the melting profile of the reference DNA samples of known methylation state, where the differences in the melting profiles are proportional to the differences in methylation state and percentage of methylated nucleotides in the DNA samples. The DNA samples may include a plurality of methylated nucleotides. In some embodiments the DNA samples may be modified by bisulfite. In other embodiments the DNA sample is fragmented by treating with restriction enzymes, physical manipulation, or chemical cleavage. The DNA samples may be isolated from biological sources or made synthetically. The methylation state of the DNA sample may indicate a change in physical conformation of the DNA. In some embodiments high resolution melting analysis is performed to determine the methylation state of DNA samples.

Additional methods are provided for measuring methyltransferase activity of an enzyme including generating defined DNA samples that are treated with a methyltransferase enzyme followed by measuring of the melting profile of the methyltransferase treated DNA samples. The amount of methylation is determined by comparing the melting profile of the methyltransferase treated DNA sample to the melting profile reference DNA sample. The methylransferase activity of the methyltransferaese enzyme is determined from the amount of methylation where the degree of methylation is proportional to the activity of the enzyme.

### DETAILED DESCRIPTION OF THE DISCLOSURE

The following disclosure describes several methods for determining the methylation state or percentage methylation of a DNA sample. Also disclosed are methods for determining the activity of methyltransferase enzymes. Kits for employing the methods are also disclosed. Several features of methods and systems in accordance with example embodiments of the invention are set forth and described in the Figures. It will be appreciated that methods and systems in accordance with other example embodiments of the invention can include additional procedures or features different than those shown in Figures. Example embodiments are described herein with respect to biological cells and DNA. However, it will be understood that these examples are for the purpose of illustrating the principals of the invention, and that the invention is not so limited.

Additionally, methods and kits in accordance with several example embodiments of the invention may not include all of the features shown in these Figures. Throughout the Figures, like reference numbers refer to similar or identical components or procedures.

Unless the context requires otherwise, throughout the specification and claims which follow, the word “comprise” and variations thereof, such as, “comnprises” and “comprising” are to be construed in an open, inclusive sense that is as “including, but not limited to.”

Reference throughout this specification to “one example” or “an example embodiment,” “one embodiment,” “an embodiment” or various combinations of these terms means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure provides methods, reagents, and kits for assaying methyltransferase activity and also for determining the total global methylation status of a sample DNA, including, but not limited to genomic DNA, using standard melting analysis as well as high-resolution melting (HRM) analysis, and like methods. One aspect of the methods is to determine methyltransferase activity accurately from high resolution melt analysis. Another aspect of the methods described in the disclosure is that the total or global methylation state of a sample DNA is determined without modification of the DNA (i.e. without bisulfite/desulfonation treatment), significantly facilitating analysis. Still another aspect of the disclosed methods is that analysis can be performed on scarce DNA samples isolated from normal, altered, pre-disease, or disease sources, using essentially any organism, including but not limited to humans, as the source of nucleic from tissues, cells, single cells, chromosomes, or specific sub-chromosomal loci without locus specific probes or assays. Further, aspects of the disclosed methods include that sample DNA may be fragmented using enzymes, physical manipulation, and chemical treatment, each used alone, or in combination, to facilitate HRM analysis. Reactions conditions utilizing specific detergents increase sensitivity allowing use of less of the sample DNA from scare clinical or cellular samples.

High Resolution Melt (HRM) analysis has been used as a sensitive micro-volume and rapid platform for assays of point mutations, genotyping, DNA fingerprinting, among other DNA characteristics, as well as for methylation analysis of PCR fragments from samples after bisulfite treatment, (Wittwer, C. T., et al. 1997; Witwer, C. T., 1997; Ririe et al., 1997; Lay et al. 1997; Reed, C. H., et al., 2004; Wojdacz, T. K., and Dobrovic A., 2007). The overall methodology is termed HRM analysis. One skilled in the art would recognize that some differences in DNA samples may be great enough that they can be detected by “regular” melt analysis and not HRM and are not limited by use of the high-resolution machines commercially available for HRM (ROCHE MOLECULAR SYSTEMS, IDAHO TECHNOLOGIES, INC. CORBETT RESEARCH CORP.).

Advances in the HRM analysis methodology utilize saturation double-strand DNA binding dyes to measure the change in fluorescence induced when double-stranded DNA melts at increasing temperature, creating characteristic melting curves that can be analyzed as melting plots to detect differences in DNA samples. This methodology is often used in conjunction with real-time PCR (Chou et al., 2005); multi-well HRM (LightScanner, IDAHO TECHNOLOGIES, INC.), HRM coupled thermal cycling devices (Lightcycler 480, ROCHE MOLECULAR SYSTEMS) or real-time rotary analysis (Rotor-Gene 6000, CORBETT RESEARCH CORP.). Additional commercially available HRM devices are available to one skilled in the art. The binding dye can also be used to measure the ratio of double- to single-stranded DNA using standard laboratory florescence monitoring equipment at a fixed temperature.

An important feature of embodiments described in the disclosure is that comparison of sample DNA-derived melting curves converted to melting plots to those from reference DNA samples of defined methylation state allows determining the relative total or global methylation state, or percentage of a particular sample DNA. In other methods, melting curves/plots from different samples with differing methylation status can be compared generating relative differences that can be used alone or in combination with comparisons to known references to assign a methylation state or assess DNA physical properties. In still other embodiments, methods allow differentiating between DNA fragments based on the DNA conformation induced by methylation, thereby differentiating the DNA samples from each other.

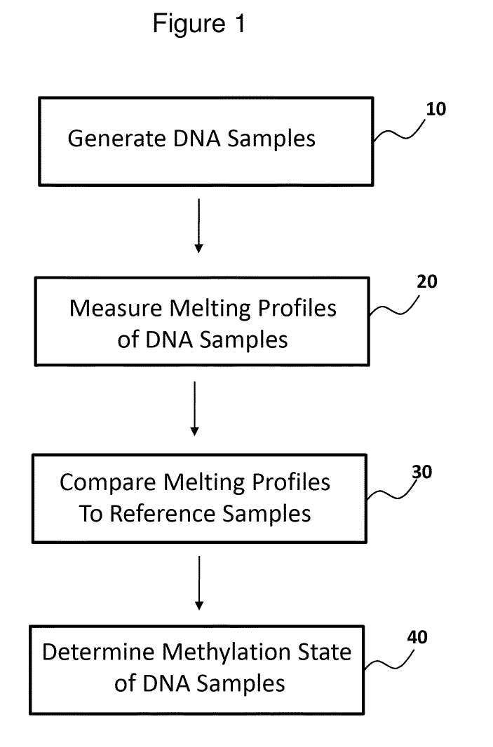

Referring now to FIG. 1, schematically shown is a method for determining the methylation state of DNA samples. Samples of DNA are generated (10), for example, by isolation or by a variety of methods, including purification using techniques known in the art that substantially purify genomic DNA from cellular contaminants, or enzymatic digestion and purification of previously isolated DNA to obtain a DNA fragment of defined size and sequence. Additionally. DNA may be generated synthetically in vitro using self assembling computationally optimized DNA assembly techniques that allow for amplification of gene sized loci amenable to manipulation and expression (CODA GENOMICS). Also both small and larger sized DNA samples can be made using standard and advanced PCR approaches. Synthetic methods offer the ability to generate standards of known methylation state using methyltransferases. Once a sample is available melting analysis can be performed that measures the melting profiles of the DNA samples (20). Comparison of melting profiles (30) of the DNA samples to reference DNA samples of known methylation state for a plurality of cytosine and adenosine nucleotides allows determining (40) of the methylation state using melting analysis techniques including HRM analysis. The differences between the melting profiles of DNA samples and the melting profiles of the known reference samples are proportional to the methylation state of the DNA sample.

A feature of these methods in certain embodiments is that analysis can be performed on DNA samples from clinically relevant biological samples that may be scarce. The ease of assaying the total methylation state of often limiting DNA samples and determining the differences in methylation states is a feature of different embodiments. Eliminating the need to modify the DNA with sodium bisulfite allows the use of lower amounts, reduces losses in yield related to chemical and physical damage induced during treatment, and reduces the time required to obtain results, which in combination permits higher sensitivity analysis. Further, the disclosed reaction conditions allow the use of large (i.e. medium or small also) sized DNA fragments that can accurately be assigned methylation states. Because the DNA is not modified and is only in single-stranded form, the output DNA after HRM analysis is suitable for use in subsequent bisulfite-based methods, or other methods known by those skilled in the art.

Sources of DNA of relatively know methylation state are used for comparison analysis and allow assignment of the total methylation state of an experimental or clinical sample. Strains of E. coli K12, including but not limited to strains deficient for methylation, mcrA/mcrBC/dam/dcm (Mcr⁻, Dam⁻, Dcm⁻) can serve as a source of non-methylated (Prokaryotic) chromosomal DNA (Woodcock, 1989; Palmer, B. R., and Marinus, M. G., 1994. Organisms cam be treated with the nucleotide analog 5-Azacytidine to demethylate the DNA as a source of low methylated DNA (Cihák A., 1974). The incorporation of 5-azacytidine into DNA (or RNA) inhibits methyltransferase enzymes causing demethylation in that sequence. Inhibition of DNA methylation occurs through the formation of stable complexes between the molecule and cellular DNA methyltransferases, thereby saturating the cells methylation machinery.

In addition, mutant human cells lines that are deficient for DNMT3b and DNMTI methyltransferase activity, can serve as a reference. In these and other similar mutant cell lines methylation is believed to be minimal, or less than about 5-10% of native levels observed in normal cell lines or tissues. It should be noted that DNA methyltransferases include a family of genes including DNMT1, DNMT3A, DNMT3B, DNMT3L, which have orthologs in many non-human species.

DNA can also be obtained from synthetic sources. PCR fragments are non-methylated by nature and can be enzymatically methylated after synthesis. Self-assembled genes are unmethylated and can be tailored for desired methylation sites (CODA GENOMICS). PCR fragments can also be synthesized in such a way so that methylated nucleoside-triphosphates (e.g. cytosine or adenosine) are substituted for their non-methylated counterparts in the reaction mixture during PCR.

The DNA isolated as non-methylated or low-methylated can be artificially methylated to high levels, and serve as reference points in HRM analysis for the determination of the relative total or global methylation status of experimental or clinical sample DNA.

Specific dyes suitable for use are in HRM methods disclosed are SYBR® Green I (MOLECULAR PROBES, INC.), SYTO® (MOLECULAR PROBES, INC.), LC Green® (IDAHO TECHNOLOGIES, INC.), and EvaGreen™ (BIOTIUM. INC.), Resolite™ (ROCHE) as well as dyes and methods disclosed in U.S. Pat. Appl. Pub. 20070020672, whose disclosure is incorporated by reference herein to the extent it aids in understanding aspects of the present invention.

In some embodiments, restriction enzymes are used to fragment genomic DNA samples into sizes suitable for high-resolution melting analysis (HRM). High resolution melting analysis on larger DNA fragments is a feature of methods described in this disclosure. Specific embodiments are performed with DNA of average size about 50 bp to about 4 kb. Other embodiments utilize DNA of average size about 4 kb to about 10 kb. Still other embodiments use sample DNA of average size about 10 kb to about 30 kb, or larger high molecular weight DNA from about 30 kb to about 300 kb. In many methods genomic DNA is fragmented into average sized fragments. More gentle isolation methods that reduce shear forces may allow isolation of larger genomic DNA fragments for analysis. These include large genomic loci or even whole chromosomes and derivatives thereof. The only limit is having an appropriate standard of known size (or known average size) and relative methylation state, or consistent reference DNA samples of specific methylation state, such that the predictability of the melting profiles allow assessment of the sample's methylation state.

Some restriction enzymes are sensitive to characteristic Eukaryotic CpG methylation, in addition to other types of methylation (e.g. cytosine, adenosine, thymidine, etc., also Dcm, Dam, Mcr, or other methylation systems). These include the commercially available and widely used class II restriction enzymes, though use of class I and class III enzymes may be performed in certain embodiments. Such restriction enzymes endonuclease activity may be blocked by CpG (^{m}CG) methylation, blocked or impaired by methylation at an enzyme's recognition site, or blocked or impaired when the methylation overlaps a recognition site. There is also a group of enzymes whose activity is blocked when the cytosine is not methylated that may be used (e.g. GlaI, BisI; SIBENZYME).

Some embodiments utilize restriction enzymes, and combinations of enzymes, where digestion of the DNA is prevented by methylation at the enzymatic recognition site, enzymes where digestion occurs at both methylated and non-methylated enzymatic recognition sites, and where digestion occurs only when the enzymatic recognition site is methylated (GlaI, BisI; SIBENZYME), as well as with methylation insensitive restriction enzymes, where there is no residue in the recognition site to be methylated (example: MseI). Such restriction enzymes include, but are not limited to the restriction enzymes or isoschizomers of: AatII; AccI; Acc651; AciI; AclI; AfeI; AgeI; AhdI; AleI; ApaI; ApaLI; AscI; AsiSI; AvaI; AvaII; BaeI; BanI; BbvCI; BceAI; BcgI; BfuAI; BglI; BmgBI; BsaI; BsaAI; BsaBI; BsaHI; BseYI; BsiEI; BsiWI; BslI; BSmAI; BsmBI; BsmFI; BspDI; BspEI; BsrBI; BsrFI; BssHII; BssKI; BstAPI; BstBI; BstUI; BStZ17I; BtgZI, Cac8I; ClaI; DraIII; DrdI; EaeI; EagI; EarI; EciI; EcoRI; EcoRV; FauI; Fnu4HI; FseI; FspI; HaeII; HgaI; HhaI; HincII; HinFI; HinP1I; HpaI; HpaII; Hpy99I; Hpy188III; HpyAV; HpyCH4IV; KasI; MboI; MluI; MmeI; MspA1I; MwoI; NaeI; NarI; NciI; NgoMIV; NheI; NlaIV; NotI; NruI; PaeR7I; PhoI; PleI; PmeI; PmlI; PshAI; PspOMI; PvuI; RsaI; RsrII; SacII; SalI; Sau3AI; Sau96I; ScrFI; SfoI; SgrAI; SmaI; SnaBI: StyD4I; TfiI; TseI; TspMI; ZraI; and any restriction enzyme that is not sensitive to CpG methylation (NEW ENGLAND BIOLABS).

Some methods employ the restriction enzymes HpaII and MspI. In other methods mixtures of 4-base and 6-base cutting enzymes are used. In still other methods the restriction enzyme McrBC is used alone or in combination with other enzymes. The restriction enzyme McrBC requires the presence of two suitably modified recognition elements appropriately spaced in the substrate (Stewart F. J., and Raleigh E. A., 1998).

In some embodiments sample DNA can be fractionated into size ranges mentioned above via physical manipulation, such as sonication, nebulization, hydrodynamic shear via circulation through narrow orifices, (e.g. HPLC pump, or passage through 28-gauge needle), or other similar physical means (Derringer, P. L., 1983; Bodentheich, et al., 1994; Oefner, et al., 1996; Davidson, 1959). In additional other embodiments, chemicals such as NaOH and like reagents may be used to fragment DNA into similar size ranges. It is also possible to modify the DNA so as to add a bulky functional group in a methylation-specific manner, which will in effect alter the melting profile with or without altering the endoniclease digestion pattern.

Additional embodiments include methods that employ combinations of fragmenting methodologies for reducing genomic DNA into suitable size ranges for HRM analysis, including but not limited to restriction enzymes, physical manipulation, and chemical treatment.

The reaction conditions for methods HRM analysis are generally known, are commercially available, and are typically done over temperature ranges of 60 to 95° C. and rising by 0.02 to 0.1° C./sec. In specific embodiments, temperature ranges of about 70° C. to about 95° C. are used rising by 0.02 to 0.05° C./sec. Commercial devices and protocols are available and have been used to address mutations, genotyping, DNA fingerprinting, and other DNA characteristics (LightScanner, IDAHO TECHNOLOGIES, INC.; Lightcycler 480, ROCHE MOLECULAR SYSTEMS; Rotor-Gene 6000, CORBETT RESEARCH CORP.).

Methods addressing improvements in the reaction conditions for high resolution melting analysis for total or global methylation determination encompass effects of dye concentrations, amount of DNA, salt effects, detergent effects, and the addition of crowding reagents (e.g. glycerol, DMSO, PEG, and Formamide) have been explored mid are known in the art.

Unique aspects of methods described in the disclosure are use of detergents from about 0.1% to about 1%, final concentration. Without being bound to a particular mode of action it is believed that detergents, including but not limited to Tween-20, Triton X-100, and Nonidet P40 act via dye containing molecules interacting with unbound dye to increase the localized concentration to a level just below the fluorescence background. When the dye component of the dye/detergent complex is bound to double-stranded DNA, then non-bound dye subunits of the same complex are near enough to the neighboring bound-dye/detergent complex to enhance the fluorescent signal. The effect is to increase the sensitivity of the fluorescent signal in melting analysis nearly 10-fold and allowing the use of about 1 ng to about 10 ng of sample DNA per measurement without increasing the background fluorescence.

The analysis of melting curve data to generate melting plots is performed with commercially available software that allows computation of first derivative (melting plots) that reveal melting transitions of the sample DNA (ROCHE MOLECULAR SYSTEMS, CORBETT RESEARCH, INC., IDAHO TECHNOLOGIES, INC.).

### A. DETERMINATION OF THE METHYLATION STATE OF ARTIFICIALLY METHYLATED PCR FRAGMENTS USING HIGH-RESOLUTION MELTING ANALYSIS

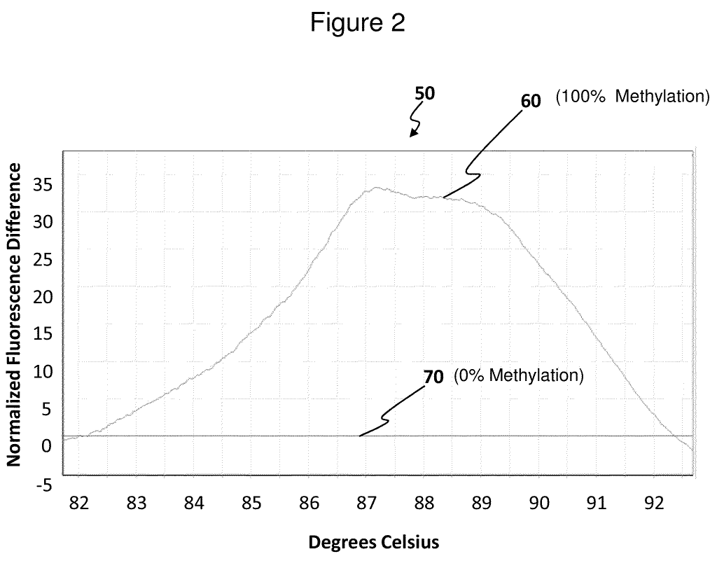

Referring now to FIG. 2, high resolution melting analysis is shown graphically for artificially methylated samples (50). HRM was performed according to the protocol in Example 1 on artificially methylated PCR fragments in order to demonstrate a proof of concept for detecting differences between differentially methylated PCR DNA fragments without bisulfite treatment. The normalized data shows the feasibility assigning an accurate methylation state or percentages to an unknown sample DNA. Primers listed in Example 1, SEQ ID No. 1-4, were use to generate 297 bp fragments that were 0% or 100% methylated, based on incubation with or without M.SssI methyltransferase. The HRM results show that the 100% methylated PCR fragment (60) has a higher fluorescence than the 0% methylated PCR fragment (70) over the temperature range of about 82.5° C. to about 92.5° C. The positive fluorescence difference indicates that there is more double-stranded DNA present in the 100% methylated sample than in the 0% methylated sample over the given temperature range. A negative fluorescence difference (not shown here) would indicate that there is more single-stranded (or melted) DNA present in 100% methylated sample than the 0% methylated sample.

The results may also be presented as a graph of their fluorescence which has been normalized to the same scale (data not shown). For this type of graph, the melting profile of the 100% methylated PCR fragment is shifted to a higher temperature than that of the 0% methylated PCR fragment. The actual melting point of the samples can also be determined from this type of graph as well, which in this case corresponds to ˜87 C for the 0% methylated fragment and ˜89 C for the 100% methylated fragment. Together, these results show that the methylation status of these two samples can be differentiated based upon their fluorescence difference at a given temperature within the range of about 82.5° C. to about 92.5° C., with 87° C. to 89° C. reflecting the maximum difference for these samples. No bisulfite treatment is necessary to visualize this difference. The results presented here suggest that any aberrantly methylated DNA sample (as found in cancer, disease, aging, and in other conditions) can be differentiated from its normal counterpart by measuring and/or comparing the melting profiles of the two DNA samples.

### B. DETERMINATION OF TOTAL METHYLATION OF THE E. COLI GENOME USING HIGH-RESOLUTION MELTING ANALYSIS

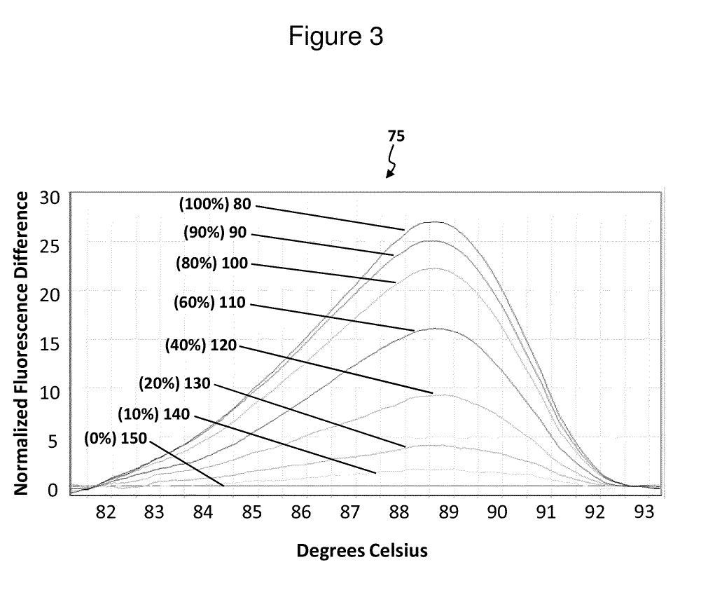

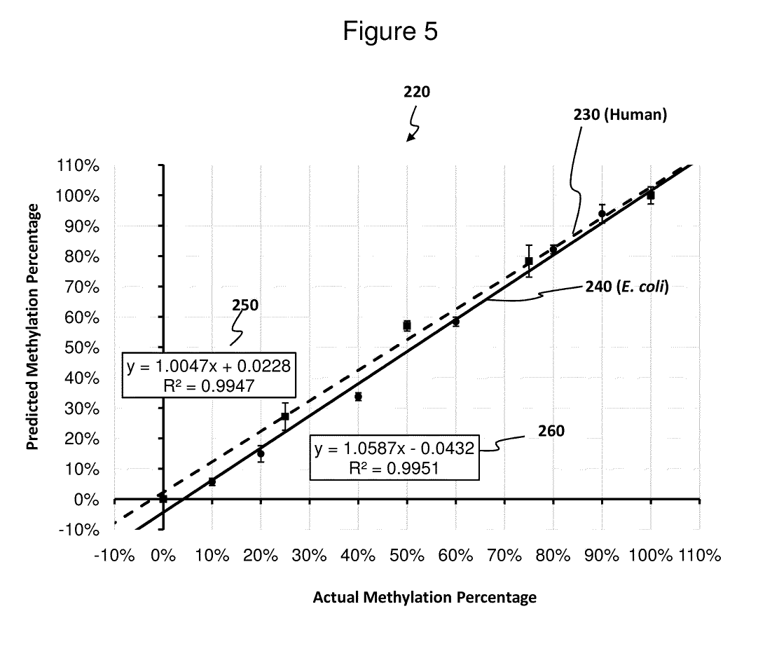

Referring now to FIG. 3, graphically shown is HRM analysis of E. coli genomic DNA (75). The chromosomal DNA from an E. coli strain that is methylation-negative was isolated and enzymatically treated with or without M.SssI methyltransferase, according to the protocol of Example 2 to generate DNA samples of specific methylation state (0% and 100%). These DNA's were mixed to generate samples with decreasing amounts of methylated DNA, from 100%, (80); 90%, (90): 80%, (100); 60%., (110); 40%, (120); 20%, (130); 10%, (140); and 0%, (150). Each was digested with HpaII restriction endonuclease, which cuts only non-methylated DNA. The HRM analysis normalized fluorescence data showed that these mixed samples displayed differences in the melting profiles that corresponded to the differences in the methylation percentage. The 0% methylated DNA sample (150), set as the baseline for comparison, has the lowest relative fluorescence within the range of 82° C. to 92° C. because it has the highest amount of fragmentation by HpaII. The average size of the enzymatically-digested DNA is small and melts at a relatively low temperature when compared to undigested DNA. Likewise, the 100% methylated DNA sample (80) has the highest relative fluorescence within the range of 82° C. to 92° C. because it has the lowest amount of fragmentation. The average-size of the DNA fragments is larger, thereby melting at a relatively higher temperature. The data also shows that DNA samples containing mixed ratios of DNA have a fluorescent profile that corresponds to the percentage of methylation in the sample (see also FIG. 5).

### C. DETERMINATION OF THE INTER-MOLECULAR METHYLATION STATE OF HUMAN DNA

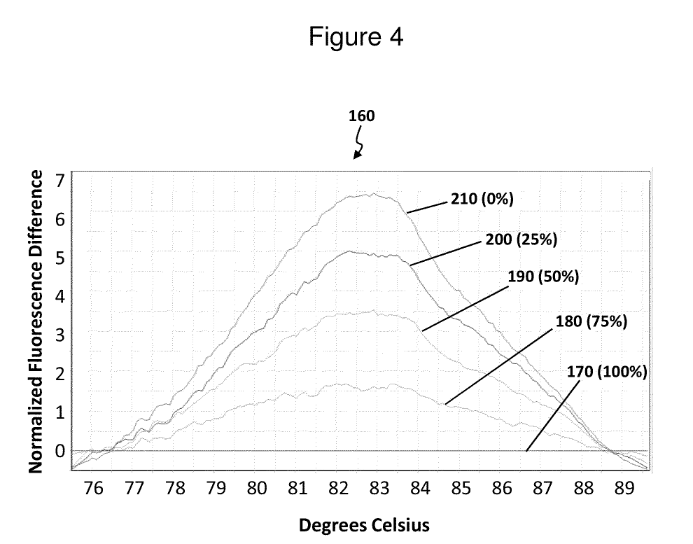

Referring now to FIG. 4, graphically shown is HRM analysis of Human derived genomic DNA (160). The genomic DNA was isolated from a cell line, which has very low levels (less than 5%) of native methylation levels (i.e. deficient for DNMT3b and DNMT1, Rhee et al. (2002). The DNA samples were enzymatically treated with or without M.SssI methyltransferase according to the protocol of Example 3 to generate DNA samples of specific methylation state (e.g. 0% and 100%). These two DNAs were mixed to generate samples with decreasing amounts of methylated DNA of 100%, (170); 75%, (180); 50%, (190); 25%, (200); and 0%, (210). Each sample was digested with McrBC restriction endonuclease, which cuts only methylated DNA. Similar to the results presented in Example 2, the HRM analysis of these mixed samples showed the differences in the melting profiles correspond to the differences in the methylation percentage. The 100% methylated DNA sample 170, set as the baseline for comparison, has the lowest relative fluorescence within the range of 76.5° C. to 86.5° C. because it has the highest amount of fragmentation by McrBC. The average size of the enzymatically-digested 100% methylated DNA is small and melts at a relatively low temperature when compared to undigested DNA. Likewise, the 0% methylated DNA sample (210) has the highest relative fluorescence because it has the lowest amount of fragmentation. The average-size of the 0% methylated DNA fragments are larger, thereby melting at a relatively higher temperature. The samples containing 25%, (200); 50%, (1190); and 75%, (180), methylation also have a fluorescence profile that corresponds to the predicted percentage of methylation in the sample (see also FIG. 5).

### D. PREDICTABILITY OF HRM FOR METHYLATION STATE

Referring now to FIG. 5, the predictability of the HRM methylation analysis methods are graphically shown for genomic DNA from human cells deficient for methylation (i.e. deficient for DNMT3b and DNMTI; FIG. 4) and for E. coli DNA (i.e. dual⁻, dcm⁻; FIG. 3) (220). The predictability of HRM determined methylation state is measured by plotting the predicted methylation percentage determined via HRM analysis against the actual methylation percentage determined from the known mixture of 0% and 100% methylated DNA samples (see FIGS. 3 and 4). The linear relationship the human DNA samples (230) (e.g. y=1.0047x+0.0228, R²=0.9947; 250) and E. coli. DNA samples (240) (e.g. y=1.0587x−0.0432, R²=0.9951; 260) can be seen. The data was examined for samples from Section C, for human DNA samples (e.g. 0%, 25%, 50%, and 100% methylation) and Section B, E. coli DNA samples (e.g. (0%, 10%, 20%, 40%, 60%, 80%, and 90%) described above, respectively. Thus HRM analysis provided a reliable method for determining methylation state of the given samples.

### E. MEASURING METHYLTRANSFERASE ACTIVITY USING HRM ANALYSIS

Methyltransferase enzymes are essential for viability and development of all organisms. The ability to assess the activity of methyltransferase enzymes using HRM would facilitate in vitro and in vivo analysis of methylation. Standard methyltransferase assays are known in the art. A prophetic assay is outline below for assaying methyltransferase activity using HRM.

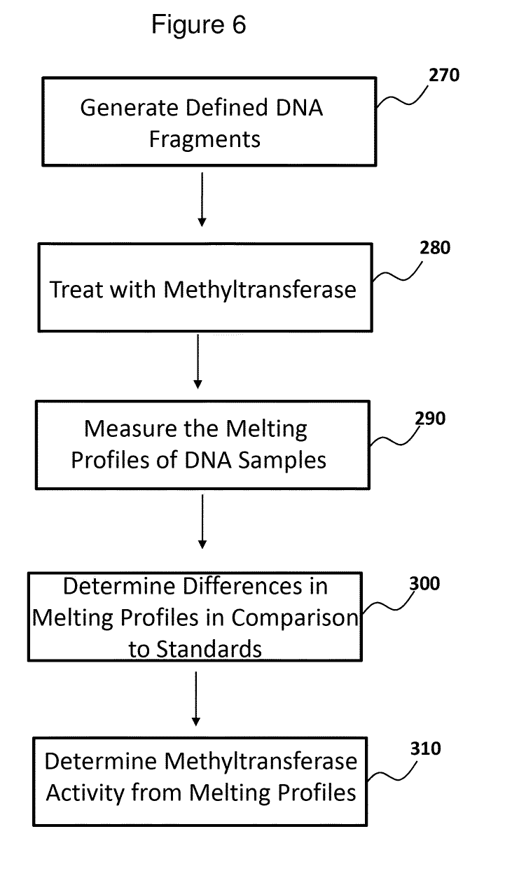

Referring now to FIG. 6, a schematic of a HRM based methyltransferase assay is shown. DNA samples could be obtained as described above and DNA fragments (270) could typically be less than 400 base pairs in size, and contain a relatively homogenous distribution of methylation sites. It is noted that the measurement of methylatransferase activity would be more accurate and precise if there is a high ratio of methylation sites to length of DNA.

Sample DNA could be a PCR product, self-assembled or synthesized, annealed set of oligonucleotides, or fragments of DNA cut from a plasmid and gel purified. Sample DNA would be divided into 3 portions (e.g. labeled A, negative control B, positive control, and C experimental).

Sample DNA would be treated with methyltransferases (280). However, no modification is required for negative, or non-methylated, control (Sample A). The positive control (Sample B) would be 100% methylated using the methyltransferase that recognizes the specific recognition sequence within the DNA. Alternatively, the positive control could be methylated during its initial synthesis, or obtained in a 100% methylated state. This is the positive, or 100% methylated, sample. It should be noted that the degree of saturation for the positive control could be verified by bisulfite treatment and sequencing, to ensure that it indeed has been 100% methylated. The experimental sample (Sample C) would be nixed with buffers, cofactors (e.g. S-adenosylmethionine, SAM), and appropriate amounts of methyltransferase on ice. To perform the methylation reaction, experimental samples would then be transferred to 37° C., or other appropriate temperature and incubate for about 1 hour, or other appropriate time. The reaction would be stopped by heating to 60° C. or similar elevated temperatures for 20 minutes or sufficient time. The reaction could also be stopped by other means (e.g. addition of detergent, proteinase, or inhibitors or by the addition of the buffer plus dye in the next step). Next, the addition of buffer and fluorescent dye that interacts with double-stranded DNA prepares the sample for HRM analysis (e.g. EvaGreen®, Resolite™. LC Green, SYTO® series, and other commercially available dyes).

Chemicals, enzymatic inhibitors, and drug candidates for therapy can be evaluated or characterized based on altered methyltransferase activity by including these compounds in the methylation reaction.

HRM can be performed as known in the art and disclosed above to measure the melting profiles of the DNA sample (290). Analysis of the HRM data could be performed as described to determine differences in the melting profiles (300) by comparison to the standards (e.g. negative and positive controls, Sample A and B). For example, by calculating the area under the melting profile curve when comparing the negative control to the positive control (compare Sample A to Sample B) and the negative control to the experimental; (compare Sample A to Sample C). The negative control is 0% methylated and is considered the baseline fluorescence. The positive control is 100% methylated. The area under the curve for the experimental sample (compare A to C) is the experimental data for the melting profile. The area under the curve for the positive control (compare A to B) is the maximum methylation percentage as indicated by the positive control. The ratio of the area under the curve for the experimental sample compared to the area under the curve for the positive control (area of Sample C/area of Sample B) is proportional to the percentage of methylation in your experimental sample, and therefore reflects the methyltransferase activity. The HRM methyltransferase assay would be useful in determining activity of known and unknown or obscure methyltransferases (310) such as from different understudied species.

The differences between methylated and non-methylated DNA samples could also be determined without measuring the entire melting profile over a temperature range, but instead by measuring fluorescence differences at a set temperature.

### F. DEFINITIONS

Scientific terms have generally been given their ordinary meaning as used in the art. The following definitions are provided for clarity:

High-Resolution Melting (HRM) Analysis: Encompasses the sensitive microliter-scale volume platforms for distinguishing DNA characteristics utilizing saturation double-stranded DNA-binding dyes and a device capable of measuring fluorescence over a temperature range combined with related software to facilitate analysis.

Reference DNA Sample: Refers to DNA of known relative methylation state or size used in HRM analysis for comparison to another reference DNA sample or experimental samples of unknown or undetermined methylation state.

Experimental DNA Sample: Refers to DNA of unknown or undetermined methylation state. The DNA can be synthetically manufactured or originating within any biological source.

Biological Source: A laboratory, clinical, pathological, forensic, or other specimen comprising an organism's tissues, fluids, cells, or sub-cellular compartments from which genomic DNA can be isolated upon which DNA methylation analysis is desired.

Total or Global Methylation Analysis: A measurement of the total number of actual methylated residues in a DNA sample among a group of the total number of possible methylated residues in a DNA sample.

DNA: DNA encompasses methylated and non-methylated single- and double-stranded DNA, unless the description or context indicated or requires otherwise. The DNA may be mixed with other unknown components, e.g. in a clinical or clinical specimen. The DNA may be chemically or enzymatically modified to the extent that it does not interfere with the underlying principles of the disclosure. The DNA may be genomic, epimeric DNA, synthetic DNA, or DNA from other sources.

Melting profile: The fluorescence recorded at each temperature over a given range of temperature, which corresponds to the ratio of double-stranded DNA to single-stranded DNA in a sample. The melting profile is often displayed as a graph with temperature on the x-axis and fluorescence on the y-axis.

Plurality of Nucleotides: more than one nucleotide in a given DNA sample.

Cytosine methylation: The presence of a methyl group at the number 5 carbon position of the pyrimidine ring of the cytosine base.

Methylation state: Whether the cytosine base at a given nucleotide position is methylated or non-methylated at the 5 carbon position of the pyrimidine ring. In the context of a plurality of nucleotides, the methylation state refers to the actual percentage of cytosine methylation among all potentially methylated cytosines in the DNA sample.

### G. UTILITY

Important features of the disclosed methods are that the total or global methylation states of DNA samples can be measured without modification of the DNA (i.e. bisulfite and desulfonation treatment). The methods thus generate superior efficiency and allow assay of samples to the nanogram range. This approach also offers an alternative for locus by locus analysis and provides relative methylation states or differentiation between DNA samples that can be used to understand general homeostasis, and the onset and diagnosis of disease. Methyltransferase activity could also be measured using the protocol outlined in this invention.

### EXAMPLES

Illustrative examples follow and are provided to better understand the disclosure. Only a few method embodiments of the disclosure have been described. Numerous modifications and variations can be made without departing from the spirit and scope of the disclosure.

### Example 1

High resolution melting (HRM) analysis was performed using a Rotor-Gene 6000 (CORBET RESEARCH, INC.) with the automatic florescence acquisition settings at a temperature from 70 to 95° C. and rising by 0.02 to 0.1° C./sec. The melting curves were normalized using the computer software included with the Rotor-Gene 6000, which allows direct comparison of samples that differ in their initial florescence levels. The software also allows graphical display of the results as a plot of differences in comparison to a reference sample (FIG. 2). All samples were run in triplicate or quadruplicate and displayed as the average florescence as determined by the software.

Determination of the methylation percentage of non-methylated and artificially-methylated PCR fragments. A 297 bp DNA fragment containing 48 double-stranded CpG dinucleotides was synthesized by PCR in a two step reaction, where PCR products generated in the first step using the oligonucleotide pairs 5′-CACAC GCGCG ATCAC CACGC GCGTG AATAC GCGCG TCCCA ACGCG CGACA TTACA TCGCG CGTGA CCTTG ACGCG CGTTA TGATG ACCCC (SEQ ID NO. 1) and 5′-ACATC ATACT TTCCA GCAAC GCGCG GAGAC TCGCG CGGTT TAAGC TCGCG CGTCC AACCT TCAGG ACGCG CGGGG TCATC ATAAC GCGCG (SEQ ID NO. 2), and 5′-GTTGC TGGAA AGTAT GATGT ATATT TGATC AATCC CCAGG GAAAA GCCTC GCGCG ATAAA GTAGA ATTGA TCGCG CGATT TGAAA AGGTC (SEQ ID NO. 3) and 5′-CCTGG ATTCG CGCGA TCTCC CAGTT CGCGC GAAGT CAAAA TCACG CGCGT CCAAT GAGAT ACGCG CGACC TTTTC AAATC GCGCG ATC (SEQ ID NO. 4) are added as templates for the second step of PCR using the primers (SEQ ID NO.'s 1 and 4). After purification, the DNA fragment was either methylated or mock-methylated using M.SssI methyltransferase (NEW ENGLAND BIOLABS), which adds a methyl group to the C5 position of all cytosine residues within the double-stranded dinucleotide recognition sequence 5′ . . . CG . . . 3′. The samples were resuspended at a concentration of 0.2 ngl/μl 50 mM NaCl, 10 mM Tris-HCl, 10 mM MgCl₂, 1 mM dithiothreitol, and 2.5 μM EvaGreen™ Dye (BIOTIUM), and High Resolution Melt analysis was performed on 10 μl using the Rotor-Gene 6000 (CORBETT RESEARCH, INC.).

### Example 2

Determination of the methylation percentage for samples that have mixed ratios of non-methylated and artificially-methylated E. coli DNA. Chromosomal DNA isolated from Escherichia coli strain which has essentially no native methylation (Mcr⁻, Dam⁻, Dcm⁻). The E. coli DNA was either methylated or mock-methylated using M.SssI methyltransferase (NEW ENGLAND BIOLABS) according to the manufacturer's instructions. After purification by phenol extraction, the two DNA samples were resuspended in 50 mM NaCl, 10 mM Tris-HCl, 10 mM MgCl₂, and 1 mM dithiothreitol at a final concentration of 10 ng/μl, resulting in a 0% and 100% methylated samples. Next, appropriate amounts of 0% and 100% methylated DNA species were mixed to achieve 100%, 90%, 80%, 60%, 40%, 20%, 10%, mid 0% methylation samples, followed by digestion with 10 units of HpaII restriction enzyme (NEW ENGLAND BIOLABS) for one hour at 37° C. (HpaII cleaves the double-stranded recognition sequence 5′ . . . CCGG . . . 3′ only when the second cytosine is non-methylated). The samples were mixed with Evagreen Dye (BIOTIUM) at a final concentration of 2.5 μM, and High Resolution Melt analysis was performed using the Rotor-Gene 6000 (Corbett Research; 100 ng of DNA in 10 μl; FIG. 2).

### Example 3

Determination of the inter-molecular methylation percentage for samples that have mixed ratios of non-methylated and artificially-methylated Human DNA. To simulate an inter-molecular methylation difference within two DNA samples, chromosomal DNA isolated from a human cell line (i.e. deficient for DNMT3b and DNMT1), which has less than 5% native methylation, was either methylated or mock-methylated using M.SssI methyltransferase (NEW ENGLAND BIOLABS) according to the manufacturer's instructions. The resulting 0% methylated and 100% methylated samples were diluted to a final concentration of 1 ng/μl and heated at 60° C. for 20 minutes to stop the methyltransferase reaction. Next, appropriate amounts of 0% and 100% methylated DNA species were mixed to achieve 100%, 75%, 50%, 25%, and 0% methylation samples, followed by digestion with 10 units of McrBC restriction enzyme (NEW ENGLAND BIOLABS) for one hour at 37° C. (McrBC cleaves DNA containing its recognition sequence only when the cytosine residue is methylated). The samples were mixed with HRM running buffer at a final concentration of 50 mM NaCl, 10 mM Tris-HCl, 10 mM MgCl₂, 1 mM dithiothreitol, 200 mM Sorbitol, 1% Tween-20, and 2.5 μM EvaGreen™ Dye (BIOTIUM), and High Resolution Melt analysis was performed in triplicate with 10 ng of DNA in 10 μl using the Rotor-Gene 6000 (CORBETT RESEARCH, INC.).

### Example 4

Kits of Parts: A description of Kit of parts and components for HRM methylation analysis would be recognized by one skilled in the art including: reagents, control DNA samples, tubes, enzymes, and instructions. Some kits could contain, buffer, double-stranded binding dye or other dyes, restriction enzymes, related classes of enzymes, spin-columns and collection tubes, software, and instructions for use.

Other kits for methyltransferase assays using HRM would supply the 0% and 100% DNA fragments (e.g. A, B, and C above), buffers, cofactors, dyes, methyltransferases, samples, software, and instructions as components of a kit of parts. The positive controls included in such kits would be validated to ensure 100% methylation.

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by specifically different equipment, and devices, and that various modifications, both as to the equipment details and operating procedures, may be accomplished without departing from the true spirit and scope of the present invention.

What is claimed is:

1. A method for determining the amount of cytosine methylation within a DNA sample, the method comprising the steps of:
generating at least one DNA sample;
measuring a melting profile of the at least one DNA sample;
comparing the melting profiles of the at least one DNA sample with a melting profile of at least one reference DNA sample of a known amount of cytosine methylation for a plurality of cytosines; and
determining the amount of cytosine methylation of the at least one DNA sample from the difference with the melting profile of the at least one reference DNA sample of known cytosine methylation amounts, wherein the differences in the melting profiles are proportional to the differences in amount of cytosine methylation of the DNA samples.

2. The method of claim 1 wherein the at least one DNA sample includes a plurality of methylated nucleotides.

3. The method of claim 1 wherein the at least one DNA sample is modified by bisulfite.

4. The method of claim 1 wherein the methylation state further comprises a change in physical conformation of the at least one DNA sample.

5. A method for measuring methyltransferase activity of an enzyme comprising the steps of:
generating at least one defined DNA sample;
treating the at least one DNA sample with at least one methyltransferase enzyme;
measuring the melting profile of the at least one methyltransferase treated DNA sample;
determining the amount of methylation by comparing the melting profile of at least one methyltransferase treated DNA sample to the melting profile of at least one reference DNA sample; and
determining the methyltransferase activity of the methyltransferase enzyme, whereby the degree of methylation is proportional to the activity of the enzyme.

6. The method of claim 5 wherein the at least one the defined DNA sample is a selected one of a methylated DNA sample and a non-methylated DNA sample.

7. A method of differentiating between DNA samples, the method comprising the steps of:
generating a set of DNA fragments from at least one reference DNA sample and from at least one experimental DNA sample;
measuring the melting profiles of the at least one reference DNA sample and of the at least one experimental sample;
comparing the melting profiles between at least two of the DNA samples; and
defining the physical state of at least one DNA sample from the differences between the melting profiles of at least two DNA samples.

8. A method for total methylation analysis of DNA isolated from a biological source, the method comprising the steps of:
treating at least one DNA sample to produce a population of DNA fragments;
generating a melting curve for the at least one population of DNA fragments;
comparing the melting curve from the at least one population of DNA fragments to the melting curve of at least one reference DNA sample of a known amount of cytosine methylation and defined average size; and
defining the total amount of cytosine methylation within the DNA isolated from a biological source from the differences between the melting profiles.

9. A method for total methylation analysis comprising the steps of:
(a) determining the melting profile of at least one reference DNA sample and of at least one experimental DNA sample by;
(b) treating the at least one reference DNA sample and at least one experimental sample to generate DNA fragments;
(c) adding fluorescent dye that binds preferentially to double stranded DNA to the at least one reference and the at least one experimental DNA sample;
(d) illuminating the at least one reference DNA sample and the at least one experimental DNA sample with a wavelength of light to generate fluorescence;
(d) measuring the fluorescence from the DNA samples as a function of DNA melting;
(e) comparing the melting profiles of at least one reference DNA sample to the melting profile of at least one of the experimental DNA samples;
(f) defining the amount of cytosine methylation of the at least one experimental DNA sample from the differences in the melting profiles between the at least one reference DNA sample and the at least one experimental DNA sample.

10. A method for total cytosine methylation analysis of DNA isolated from a biological source, the method comprising the steps of:
isolating at least one experimental DNA sample from a biological source;
treating the at least one experimental DNA sample to generate a defined average size;
measuring the melting profiles of at least one reference DNA sample of a known amount of cytosine methylation and defined average size;
measuring the melting profile of at least one experimental DNA sample of defined average size;
comparing the melting profiles between at least one of the reference DNA samples and at least one of the experimental DNA samples; and
defining the amount of cytosine methylation of the at least one experimental DNA sample from the differences between the melting profiles of the at least one reference DNA sample and the at least one experimental DNA sample.

11. The method of claim 1, wherein the DNA sample has an average size range that is selected from the group consisting of 50 bp to 4.0 kb, 4 kb to 10 kb, 10 kb to 30 kb, and 30 kb to 300 kb.

12. The method of claim 1, wherein the DNA sample is generated by a treatment selected from the group consisting of digesting with at least one restriction enzyme, physical manipulation, and chemical cleavage.

13. The method of claim 1 wherein the digestion by the at least one restriction enzyme further comprises recognition of a recognition site selected from the group consisting of a recognition site without a methylated or unmethylated CpG dinucleotide, a recognition site where digestion by the at least one enzyme is prevented by methylation at the enzymatic recognition site, a recognition site where digestion by at least one of the enzymes occurs at both methylated and non-methylated enzymatic recognition site, and a recognition site where digestion by at least one of the enzymes occurs only when the enzymatic recognition site is methylated.

14. The method of claim 12, further comprising digesting with at least one restriction enzyme chosen from the group consisting of AatII: AccI; Acc651; AciI; AclI: AfeI: AgeI; AhdI; AleI; ApaI; ApaLI; AscI; AsiSI; AvaI; AvaII; BaeI; BanI; BbvCI; BceAI; BcgI; BfuAI; BglI; BisI; BmgBI; BsaI; BsaAI; BsaBI; BsaHI; BseYI; BsiEI; BsiWI; BslI; BSmAI; BsmBI; BsmFI; BspDI; BspEI; BsrBI; BsrFI; BssHII; BssKI; BstAPI; BstBI; BstUI; BStZ17I; BtgZI; Cac8I; ClaI; DraIII; DrdI; EaeI; EagI; Earl; EciI; EcoRI; EcoRV; FauI; Fnu4HI; FseI; FspI; GlaI; HaeII; HgaI; HhaI; HincII; HinFI; HinP1I; HpaI; HpaII; Hpy99I; Hpy1881II; HpyAV; HpyCH4IV; KasI; MboI; McrBC; MluI; MmeI; MspA1I; MwoI; NaeI; NarI; NciI; NgoMIV; NheI; NlaIV; NotI; NruI; PaeR7I; PhoI; PleI; PmeI; PmlI; PshAI; PspOMI; PvuI; RsaI; RsrII; SacII; SalI; Sau3AI; Sau96I; ScrFI; SfoI; SgrAI; SmaI; SnaBI: StyD4I; TfiI; TseI; TspMI; ZraI, and isoschizomers.

15. The method of claim 14, wherein the at least one restriction enzyme is McrBC.

16. The method of claim 1 wherein the melting analysis is performed using a device selected from the group consisting of a rotary high resolution melt device, a thermocycler, standard resolution melt device, a multi-well plate-based high resolution devices, and a high resolution melt thermocycler device.

17. The method of claim 1 wherein the melting profiles are performed using high resolution melt analysis.

18. The method of claim 1, wherein the amount of cytosine methylation of at least one DNA sample correlates with a known amount of cytosine methylation of a disease and susceptibility to the onset of a disease.

19. The method of claim 1, wherein the melting profiles are performed in the presence of detergent, wherein the sensitivity of the fluorescence signal is increased.

20. A kit of parts comprising a buffer, a double stranded binding dye, non-methylated and pre-methylated standards, restriction enzymes, modifying enzymes, spin-columns, collection tubes, and instructions for use.

21. A kit of claim 20 further comprising a computer software program for analysis of melting profiles.

22. A kit of claim 20 further comprising a device for physical modification of DNA.
